(19) United States
(12) Patent Application Publication
(10) Pub. No.: US 2015/0352031 A1
(43) Pub. Date: Dec. 10, 2015

(54) COMPOSITION

(71) Applicant: CONOPCO, INC., D/B/A UNILEVER, Englewood Cliffs, NJ (US)

(72) Inventors: Stephen Norman BATCHELOR, Chester (GB); Adam John LIMER, Northwich (GB); Xuezhi TANG, Shanghai (CN)

(73) Assignee: Conopco, Inc., d/b/a UNILEVER, Englewood Cliffs, NJ (US)

(21) Appl. No.: 14/760,628

(22) PCT Filed: Jan. 3, 2014
(86) PCT No.: PCT/EP2014/050059

§ 371 Date: Jul. 13, 2015

(30) Foreign Application Priority Data
Jan. 15, 2013 (CN) PCT/CN2013/070479

(51) Int. Cl. A61K 8/84; A61Q 5/10

(52) U.S. Cl. CPC A61K 8/84; A61Q 5/10; A61K 2800/4324

(57) ABSTRACT

A hair colouring composition comprising an arylated polyethyleneimine dye wherein the arylated polyethyleneimine dye comprises a polyethyleneimine having covalently bound to the nitrogen groups of the polyethyleneimine: a. a negatively charged reactive dye and; b. aryl group or substituted aryl group; c. in which the aryl group or substituted aryl group and the negatively charged reactive dye are different moieties.

The present invention relates to arylated polyethleneimine dyes in hair care colouring compositions

U.S. Pat. No. 4,182,612 (Gillette) discloses polyethyleneimine cationic polymers covalently linked to dyes for the colouration of hair. The dyes used do not carry negatively charged substituents.

WO2011/113680 (Unilever) discloses polyethylene and polypropylene imines bound to reactive dyes for the colouration of hair.

However, there remains the need to increase the substantivity of dye-polymers to hair and to reduce skin staining

### SUMMARY OF THE INVENTION

The present invention relates to a hair colouring composition comprising an arylated polyethyleneimine dye in which the arylated polyethyleneimine dye comprises a polyethyleneimine having covalently bound to the nitrogen groups of the polyethyleneimine:

(i) a negatively charged reactive dye and;

(ii) an aryl group or substituted aryl group;

in which the aryl group or substituted aryl group and the negatively charged reactive dye are different moieties.

In the context of the present invention the dye carries a negatively charged group before and after addition to the polyethyleneimine.

In a second aspect the invention relates to a method of colouring hair comprising the step of applying the above composition to the hair.

In a third aspect the invention provides the use of the above mentioned arylated polyethylenimine dye for colouring hair.

In a fourth aspect the invention relates to a polyethyleneimine having covalently bound to the nitrogen groups of the polyethyleneimine:

(i) a negatively charged reactive dye and;

(ii) aryl group or substituted aryl group;

in which the aryl group or substituted aryl group and the negatively charged reactive dye are different moieties.

### DETAILED DESCRIPTION OF THE INVENTION

Unless specified otherwise, all wt % values quoted hereinafter are percentages by weight based on total weight of the hair treatment composition.

Polyethyleneimine has been abbreviated in the text to PEI.

Dye Polymer

Arylated polyethyleneimines are polyethyleneimines, where a single or a number of amine functions are reacted with one or more aryl containing moieties to form a side chain containing an aryl group on the polymer. Arylation is the covalent addition of an organic group containing an aryl group to the polymer. The preferred aryl groups are napthyl and phenyl, most preferably phenyl.

Preferably the arylation of the polymer is the reaction of an aryl compounding bearing an epoxide side group with the polyethyleneimine. Preferred epoxide compounds are selected from

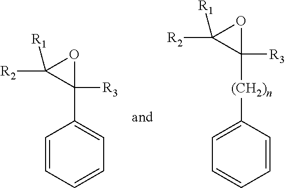

Where R₁ R₂ and R₃ are independently selected from H, C1-C6 branched, cyclic or linear alkyl. Preferably R₁ is H, more preferably R₁ and R₂ are H, most preferably and R₁ R₂ and R₃ are H, n is from 1 to 4. The phenyl ring may be further substituted by organic groups, preferably uncharged organic groups. Preferably CH₃, C2-C6 branched, cyclic or linear alkyl.

The epoxide may be prepared by epoxidation of the corresponding alkene using a peracid. Suitable alkenes include styrene, alpha-methyl styrene, trans-beta-methyl-styrene, allylbenzene, 4-phenyl-1-butene, 3-methyl-1-phenyl-1-propene, 2,3-dibenzyl-1,3-butadiene, 1,2-dihydronaphthalene, alpha-2-dimethyl styrene, 4-methyl styrene, divinyl benzene, 4-tert-butyl benzene, 2,4-dimethyl styrene.

A reaction scheme is exemplified below:

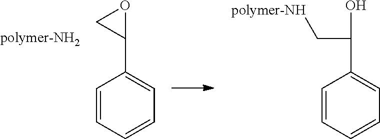

The polymer may be arylated before or after the covalent addition of the reactive dye, preferably before.

Alternatively the arylation of the polymer is achieved by the reaction of an aryl compounding bearing a carboxylic acid group with the polyalkyl amine. Preferred acid compounds are selected from

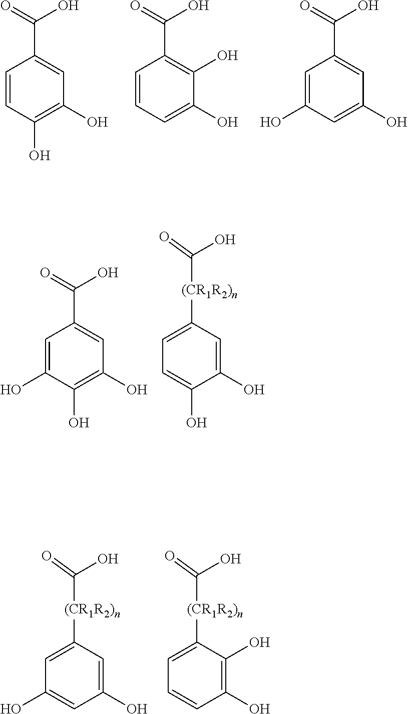

Where R₁ and R₂ are independently selected from H, OH, NH2, NR₁R₂, C1-C6 branched, cyclic or linear alkyl. Preferably R₁ is H, more preferably R₁ and R₂ are H. n is from 1 to 4. The phenyl ring may be further substituted by organic groups, preferably uncharged organic groups. Preferably CH₃, C2-C6 branched, cyclic or linear alkyl.

A reaction scheme is exemplified below:

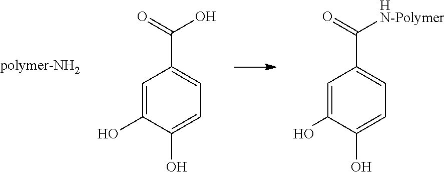

The polymer may be arylated before or after the covalent addition of the reactive dye, preferably before.

Preferably the substituted aryl group of the arylated polyethyleneimine dye has the structure:

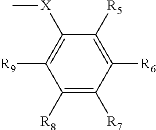

In which X covalently connects to a nitrogen of the PEI and is preferably selected from a hydroxy amide or amide group, more preferably —CH₂—CH(OH)— or an acid amide group (—NHOC)—; R₅, R₆, R₇, R₈, R₉ are independently selected from H, C1-C1 alkyl or hydroxy groups, such that at least two R₁ to R₅ groups are equal to H.

Preferably the aryl group of the arylated polyethyleneimine dye is phenyl

Example of structures are:

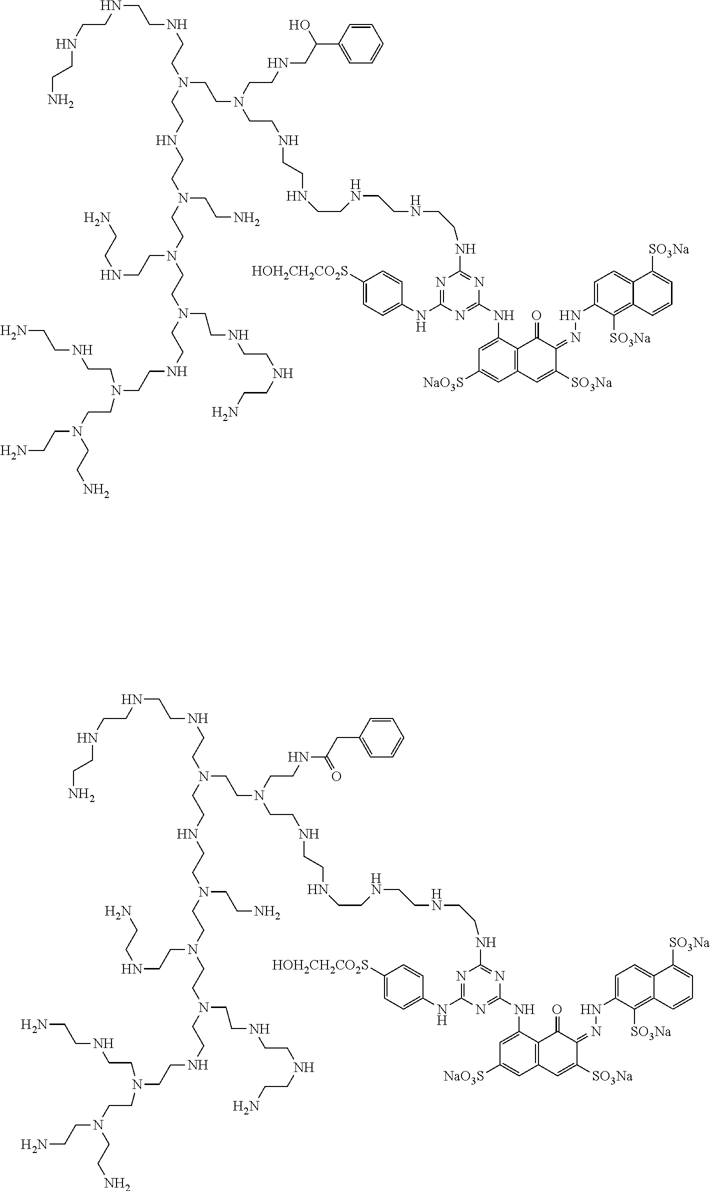

Polymer Before Arylation and Covalent Attachment to the Dye

The polyamine polymer comprises at least two free —NH or —NH₂ group for reaction. More preferably, the polymer contains at least four —NH or —NH₂ groups, most preferably at least six —NH or —NH₂ group. Examples of such polymers are polyethyleneimine, polypropylene-imine, polyvinylamine; polyvinylimine; aminosilcone.

Preferably, the polymers have an overall cationic charged at pH 5.0. Preferably, the polymer comprises quaternary amine groups or tertiary amines.

Poly amines are generally linear or branched. The polyethyleneimine comprises primary, secondary or tertiary amines or a mixture thereof.

The arylated polyamines polymer before binding to the dye preferably has a weight average molecular weight of 300-60000, more preferably 300-2000, most preferably 500-1300. The polymer may be branched or linear, preferably branched.

The molecular weights are determined by dynamic light scattering using a Zetasizer Nano (Malvern).

Preferably the PEI before reaction with the aryl moieties contains at least 4 primary or secondary amines.

Preferably PEI before reaction has from 6 to 100 nitrogen atoms, more preferably from 12 to 40 nitrogen atoms, most preferably from 18 to 36.

Specific examples of polyethylene imines are PEI-300, PEI-500, PEI-600, PEI-700, PEI-800, PEI-1000, PEI-1500, PEI-1800, PEI-2000, PEI-2500, PEI-5000, PEI-10000, PEI-25000, PEI 50000 and PEI-70000, wherein the integer represents the number average molecular weight (M_{w}) of the polymer. PEI's which are designated as such are available through Aldrich.

Preferably the PEI is not alkoxylated.

Suitable reactive dyes for use with PEI are described in Industrial Dyes (K. Hunger ed, Wiley VCH 2003). Many Reactive dyes are listed in the colour index (Society of Dyers and Colourists and American Association of Textile Chemists and Colorists).

Reactive groups are preferably selected from heterocyclic reactive groups and, a sulfooxyethylsulfonyl reactive group (—SO₂CH₂CH₂OSO₃Na), which is converts to a vinylsulfone in alkali. The heterocyclic reactive groups are preferably nitrogen contains aromatic rings bound to a halogen or an ammonium group or a quaternary ammonium group which react with NH₂ or NH groups of the polymers to form a covalent bonds. The halogen is preferred, most preferably Cl or F.

Preferably, the reactive dye comprises a reactive group selected from dichlorotriazinyl, difluorochloropyrimidine, monofluorotrazinyl, dichloroquinoxaline, vinylsulfone, difluorotriazine, monochlorotriazinyl, bromoacrlyamide and trichloropyrimidine.

Preferably the dye is sulphonated.

The reactive group may be linked to the dye chromophore via an alkyl spacer for example: dye-NH—CH₂CH₂-reactive group.

Especially preferred heterocylic reactive groups are

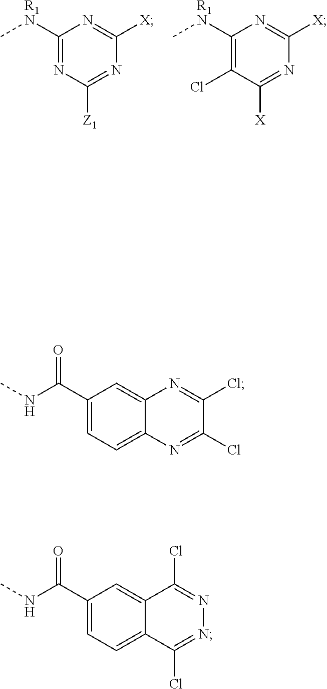

Wherein R₁ is selected from H or alkyl, preferably H.

X is selected from F or Cl

When X═Cl, Z₁ is selected from —Cl, —NR₂R₃; —OR₂, —SO₃Na

When X═F, Z₁ is selected from —NR₂R₃

R₂ and R₃ are independently selected from H, alkyl and aryl groups. Aryl groups are preferably phenyl and are preferably substituted by —SO₃Na or —SO₂CH₂CH₂OSO₃Na.

The phenyl groups may be further substituted with suitable uncharged organic groups, preferably with a molecular weight lower than 200. Preferred groups include —CH₃, —C₂H₅, and —OCH₃.

The alkyl groups may be further substituted with suitable uncharged organic groups, preferably with a molecular weight lower than 200. Preferred groups include —CH₃, —C₂H₅, —OH, —OCH₃, —OC₂H₄OH.

Most preferred heterocyclic reactive groups are selected from

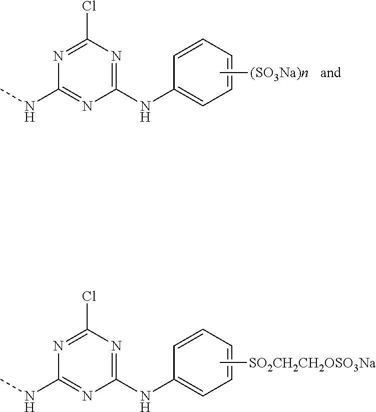

Where n=1 or 2, preferably 1.

Preferably the reactive dye contains more than one reactive group, preferably two or three.

Preferably, the reactive dye comprises a chromophore selected from azo, anthraquinone, phthalocyanine, formazan and triphendioaxazine.

Where the dye is an azo dye it is preferred that the azo dye is not an azo-metal complex dye.

Preferably the reaction of the polyamine and the reactive dye to form the dye polymers, takes place in water at alkaline pH, preferable pH=10 to 11.5, at temperature of 40-100° C. for 1 to 3 hours after the dye is added to the solution. Thereafter the solution is cooled to room temperature and neutralised to pH=7 within 1 to 2 hours. The level of polyamine in the reaction solution is preferable from 2 to 50 wt %, more preferably from 5 to 20 wt %. These conditions minimise the production of hydrolysed dye.

Examples of reactive dyes include reactive black 5, reactive blue 19, reactive red 2, reactive blue 171, reactive blue 269, reactive blue 11, reactive yellow 17, reactive, reactive orange 4, reactive orange 16, reactive green 19, reactive brown 2, reactive brown 50.

Reactive blue dyes are preferably selected from anthraquinone, mono azo, bis-azo, triphenodioxazine, and phthalocyanine, more preferably anthraquinone, bis-azo, and triphenodioxazine, most preferably bis-azo and triphenodioxazine.

A preferred blue bis-azo dye is of the form

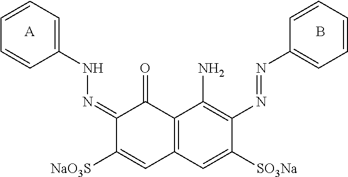

Where one or both of the A and B rings are substituted by a reactive group. The A and B rings may be further substituted by sulphonate groups (SO₃Na). The A and B rings may be further substituted with suitable uncharged organic groups, preferably with a molecular weight lower than 200. Preferred uncharged organic groups are —CH₃, —C₂H₅, and —OCH₃.

Preferred blue bis-azo dye dyes are Reactive Black 5, Reactive Blue 171, Reactive Blue 154, Reactive Blue 184, Reactive Blue 207, Reactive Blue 214, Reactive Blue 217, Reactive Blue 203, Reactive Blue 225, Levafix Navy CA, Procion Navy H-EXL, Reactive Blue 176, Reactive Blue 109, Reactive Blue 230, Reactive Blue 225, Reactive Blue 222, Reactive Blue 250 and Reactive Blue 281.

A preferred blue anthraquinone dye is of the form:

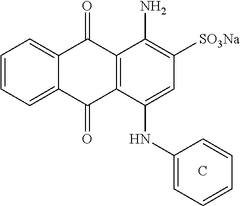

Where the C ring is substituted by a reactive group. The dye may be further substituted with sulphonate groups (SO₃Na) and suitable uncharged organic groups, preferably with a molecular weight lower than 200. Preferred uncharged organic groups are —CH₃, —C₂H₅, and —OCH₃.

A preferred blue triphenodioxazine dye is of the form:

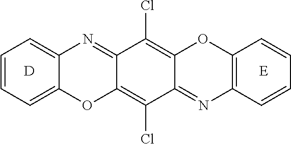

Where the D and E rings are substituted by a reactive groups. Preferably the D and E rings are further substituted by sulphonate groups (SO₃Na).

Examples of reactive blue dyes are reactive blue 2, reactive blue 4, reactive blue 5, reactive blue 7, reactive blue 15, reactive blue 19, reactive blue 27, reactive blue 29, reactive blue 49, reactive blue 50, reactive blue 74, reactive blue 94, reactive blue 246, reactive blue 247, reactive blue 247, reactive blue 166, reactive blue 109, reactive blue 187, reactive blue 213, reactive blue 225, reactive blue 238, reactive blue 256. Further structures are exemplified below:

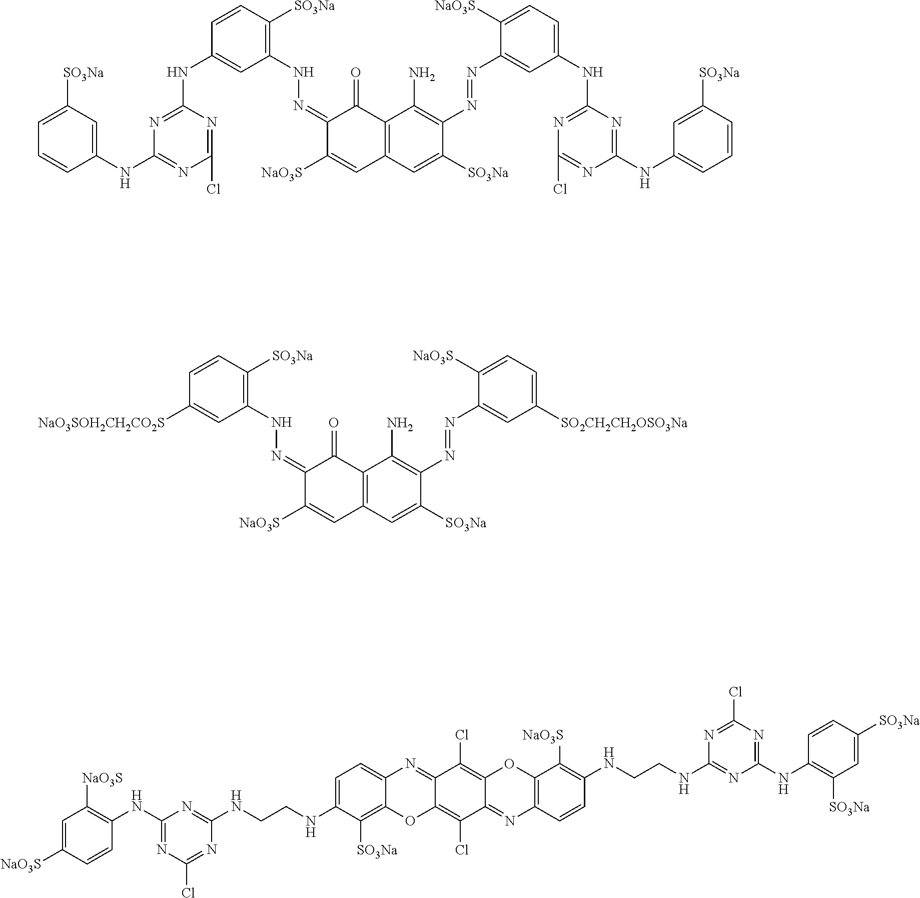

Reactive Red dyes are preferably selected from mono-azo and bis-azo dyes.

A preferred reactive red azo dye is of the form:

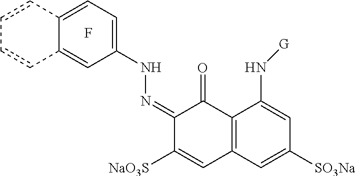

Where the F ring is optionally extended to form a naphthyl group and is optionally substituted by groups selected from sulphonate groups (SO₃Na) and a reactive group.

G is selected from a reactive group, H, or alky group. A reactive group must be present on the dye.

Examples of reactive red dyes are reactive red 2, reactive red 3, reactive red 4, reactive red 8, reactive red 9, reactive red 12, reactive red 13, reactive red 17, reactive red 22, reactive red 24, reactive red 29, reactive red 33 reactive red 139, reactive red 198 and reactive red 141.

Reactive yellow and orange dyes are preferably selected from mono-azo dyes. Examples of reactive yellow and orange dyes are reactive yellow 1, reactive yellow 2, reactive yellow 3, reactive yellow 16, reactive yellow 17, reactive yellow 25, reactive yellow 39, reactive orange 107, reactive yellow 176 and reactive yellow 135.

Combination of reactive dyes may be used to obtain a wide colour palette with use of a limited number of dyes. Preferably, a trichromate system consisting of a mixture of three reactive dyes. Preferably, the trichromate system contains a combination of a reactive blue or a reactive black dye, a reactive red and a reactive yellow dye. For example reactive black 5, reactive yellow 176 and reactive red 239; reactive blue 171, reactive yellow 176 and reactive red 141.

Preferably the mole ratio of PEI to dye is from 1:1 to 2:1.

Preferably, the dye polymer is obtainable by reacting the polymer with from 0.1 to 40 wt % reactive dye, most preferably from 5 to 20 wt %.

Composition

It is preferable that the hair colourants of the present application are formulated as conditioning compositions.

Preferably, the water used to formulate all compositions has a French hardness of from 0 to 36 degrees, more preferably 0 to 24 degrees, most preferably from 0 to 2 degrees.

Preferably, the water used to formulate all compositions contains less than 1 ppm of chlorine based bleaching agents such as chlorine dioxide or hypochlorite. Most preferably less than 50 ppb.

Silicone Conditioning Agents

The compositions of the invention can contain, emulsified droplets of a silicone conditioning agent, for enhancing conditioning performance.

Suitable silicones include polydiorganosiloxanes, in particular polydimethylsiloxanes which have the CTFA designation dimethicone. Also suitable for use compositions of the invention (particularly shampoos and conditioners) are polydimethyl siloxanes having hydroxyl end groups, which have the CTFA designation dimethiconol. Also suitable for use in compositions of the invention are silicone gums having a slight degree of cross-linking, as are described for example in WO 96/31188.

The viscosity of the emulsified silicone itself (not the emulsion or the final hair conditioning composition) is typically at least 10,000 cst at 25° C. the viscosity of the silicone itself is preferably at least 60,000 cst, most preferably at least 500,000 cst, ideally at least 1,000,000 cst. Preferably the viscosity does not exceed 10⁹ cst for ease of formulation.

Emulsified silicones for use in the shampoo compositions of the invention will typically have an average silicone droplet size in the composition of less than 30, preferably less than 20, more preferably less than 10 μm, ideally from 0.01 to 1 μm. Silicone emulsions having an average silicone droplet size of ≦0.15 μm are generally termed microemulsions.

Emulsified silicones for use in the conditioner compositions of the invention will typically have an size in the composition of less than 30, preferably less than 20, more preferably less than 15. Preferably, the average silicone droplet is greater than 0.5 μm, more preferably greater than 1 μm, ideally from 2 to 8 μm.

Silicone particle size may be measured by means of a laser light scattering technique, for example using a 2600D Particle Sizer from Malvern Instruments.

Examples of suitable pre-formed emulsions include Xiameter MEM 1785 and microemulsion DC2-1865 available from Dow Corning. These are emulsions/microemulsions of dimethiconol. Cross-linked silicone gums are also available in a pre-emulsified form, which is advantageous for ease of formulation

A further preferred class of silicones for inclusion in shampoos and conditioners of the invention are amino functional silicones. By “amino functional silicone” is meant a silicone containing at least one primary, secondary or tertiary amine group, or a quaternary ammonium group. Examples of suitable amino functional silicones include: polysiloxanes having the CTFA designation “amodimethicone”. Specific examples of amino functional silicones suitable for use in the invention are the aminosilicone oils DC2-8220, DC2-8166 and DC2-8566 (all ex Dow Corning).

Suitable quaternary silicone polymers are described in EP-A-0 530 974.

A preferred quaternary silicone polymer is K3474, ex Goldschmidt.

Also suitable are emulsions of amino functional silicone oils with non ionic and/or cationic surfactant. Pre-formed emulsions of amino functional silicone are also available from suppliers of silicone oils such as Dow Corning and General Electric. Specific examples include DC939 Cationic Emulsion and the non-ionic emulsions DC2-7224, DC2-8467, DC2-8177 and DC2-8154 (all ex Dow Corning).

With some shampoos it is preferred to use a combination of amino and non amino functional silicones

The total amount of silicone is preferably from 0.01 wt % to 10% wt of the total composition more preferably from 0.1 wt % to 5 wt %, most preferably 0.5 wt % to 3 wt % is a suitable level.

Non-Silicone Oily Conditioning Components

Compositions according to the present invention may also comprise a dispersed, non-volatile, water-insoluble oily conditioning agent. By “insoluble” is meant that the material is not soluble in water (distilled or equivalent) at a concentration of 0.1% (w/w), at 25° C.

Suitable oily or fatty materials are selected from hydrocarbon oils, fatty esters and mixtures thereof. Straight chain hydrocarbon oils will preferably contain from about 12 to about 30 carbon atoms. Also suitable are polymeric hydrocarbons of alkenyl monomers, such as C₂-C₆ alkenyl monomers.

Specific examples of suitable hydrocarbon oils include paraffin oil, mineral oil, saturated and unsaturated dodecane, saturated and unsaturated tridecane, saturated and unsaturated tetradecane, saturated and unsaturated pentadecane, saturated and unsaturated hexadecane, and mixtures thereof. Branched-chain isomers of these compounds, as well as of higher chain length hydrocarbons, can also be used.

Suitable fatty esters are characterised by having at least 10 carbon atoms, and include esters with hydrocarbyl chains derived from fatty acids or alcohols. Monocarboxylic acid esters include esters of alcohols and/or acids of the formula R′COOR in which R′ and R independently denote alkyl or alkenyl radicals and the sum of carbon atoms in R′ and R is at least 10, preferably at least 20. Di- and trialkyl and alkenyl esters of carboxylic acids can also be used.

Particularly preferred fatty esters are mono-, di- and triglycerides, more specifically the mono-, di-, and tri-esters of glycerol and long chain carboxylic acids such as C₁-C₂₂ carboxylic acids. Preferred materials include cocoa butter, palm stearin, sunflower oil, soyabean oil and coconut oil.

The oily or fatty material is suitably present at a level of from 0.05 wt % to 10 wt %, preferably from 0.2 wt % to 5 wt %, more preferably from about 0.5 wt % to 3 wt %.

Cationic Conditioning Surfactants

Preferably the composition comprises a cationic surfactant.

Suitable conditioner compositions will typically comprise one or more conditioning surfactants which are cosmetically acceptable and suitable for topical application to the hair.

Suitable conditioning surfactants include those selected from cationic surfactants, used singly or in admixture. Preferably, the cationic surfactants have the formula N⁺R¹R²R³R⁴ wherein R¹, R², R³ and R⁴ are independently (C₁ to C₃₀) alkyl or benzyl. Preferably, one, two or three of R¹, R², R³ and R⁴ are independently (C₄ to C₃₀) alkyl and the other R¹, R², R³ and R⁴ group or groups are (C₁-C₆) alkyl or benzyl. More preferably, one or two of R¹, R², R³ and R⁴ are independently (C₆ to C₃₀) alkyl and the other R¹, R², R³ and R⁴ groups are (C₁-C₆) alkyl or benzyl groups. Optionally, the alkyl groups may comprise one or more ester (—OCO— or —COO—) and/or ether (—O—) linkages within the alkyl chain. Alkyl groups may optionally be substituted with one or more hydroxyl groups. Alkyl groups may be straight chain or branched and, for alkyl groups having 3 or more carbon atoms, cyclic. The alkyl groups may be saturated or may contain one or more carbon-carbon double bonds (eg, oleyl). Alkyl groups are optionally ethoxylated on the alkyl chain with one or more ethyleneoxy groups.

Suitable cationic surfactants for use in conditioner compositions according to the invention include cetyltrimethylammonium chloride, behenyltrimethylammonium chloride, cetylpyridinium chloride, tetramethylammonium chloride, tetraethylammonium chloride, octyltrimethylammonium chloride, dodecyltrimethylammonium chloride, hexadecyltrimethylammonium chloride, octyldimethylbenzylammonium chloride, decyldimethylbenzylammonium chloride, stearyldimethylbenzylammonium chloride, didodecyldimethylammonium chloride, dioctadecyldimethylammonium chloride, tallowtrimethylammonium chloride, dihydrogenated tallow dimethyl ammonium chloride (eg, Arquad 2HT/75 from Akzo Nobel), cocotrimethylammonium chloride, PEG-2-oleammonium chloride and the corresponding hydroxides thereof. Further suitable cationic surfactants include those materials having the CTFA designations Quaternium-5, Quaternium-31 and Quaternium-18. Mixtures of any of the foregoing materials may also be suitable. A particularly useful cationic surfactant for use in conditioners according to the invention is cetyltrimethylammonium chloride, available commercially, for example as GENAMIN CTAC, ex Hoechst Celanese. Another particularly useful cationic surfactant for use in conditioners according to the invention is behenyltrimethylammonium chloride, available commercially, for example as GENAMIN KDMP, ex Clariant.

Another example of a class of suitable cationic surfactants for use in the invention, either alone or together with one or more other cationic surfactants, is a combination of (i) and (ii) below:

(i) an amidoamine corresponding to the general formula (I):

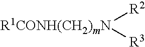

in which R¹ is a hydrocarbyl chain having 10 or more carbon atoms,
R² and R³ are independently selected from hydrocarbyl chains of from 1 to 10 carbon atoms, and m is an integer from 1 to about 10; and

(ii) an acid.

As used herein, the term hydrocarbyl chain means an alkyl or alkenyl chain.

Preferred amidoamine compounds are those corresponding to formula (I) in which

R¹ is a hydrocarbyl residue having from about 11 to about 24 carbon atoms,

R² and R³ are each independently hydrocarbyl residues, preferably alkyl groups, having from 1 to about 4 carbon atoms, and m is an integer from 1 to about 4.

Preferably, R² and R³ are methyl or ethyl groups.

Preferably, m is 2 or 3, i.e. an ethylene or propylene group.

Preferred amidoamines useful herein include stearamido-propyldimethylamine, stearamidopropyldiethylamine, stearamidoethyldiethylamine, stearamidoethyldimethylamine, palmitamidopropyldimethylamine, palmitamidopropyl-diethylamine, palmitamidoethyldiethylamine, palmitamidoethyldimethylamine, behenamidopropyldimethyl-amine, behenamidopropyldiethylmine, behenamidoethyldiethyl-amine, behenamidoethyldimethylamine, arachidamidopropyl-dimethylamine, arachidamidopropyldiethylamine, arachid-amidoethyldiethylamine, arachidamidoethyldimethylamine, and mixtures thereof.

Particularly preferred amidoamines useful herein are stearamidopropyldimethylamine, stearamidoethyldiethylamine, and mixtures thereof.

Commercially available amidoamines useful herein include: stearamidopropyldimethylamine with tradenames LEXAMINE S-13 available from Inolex (Philadelphia Pa., USA) and AMIDOAMINE MSP available from Nikko (Tokyo, Japan), stearamidoethyldiethylamine with a tradename AMIDOAMINE S available from Nikko, behenamidopropyldimethylamine with a tradename INCROMINE BB available from Croda (North Humberside, England), and various amidoamines with tradenames SCHERCODINE series available from Scher (Clifton N.J., USA).

A protonating acid may be present. Acid may be any organic or mineral acid which is capable of protonating the amidoamine in the conditioner composition. Suitable acids useful herein include hydrochloric acid, acetic acid, tartaric acid, fumaric acid, lactic acid, malic acid, succinic acid, and mixtures thereof. Preferably, the acid is selected from the group consisting of acetic acid, tartaric acid, hydrochloric acid, fumaric acid, lactic acid and mixtures thereof.

The primary role of the acid is to protonate the amidoamine in the hair treatment composition thus forming a tertiary amine salt (TAS) in situ in the hair treatment composition. The TAS in effect is a non-permanent quaternary ammonium or pseudo-quaternary ammonium cationic surfactant.

Suitably, the acid is included in a sufficient amount to protonate more than 95 mole % (293 K) of the amidoamine present.

In conditioners of the invention, the level of cationic surfactant will generally range from 0.01% to 10%, more preferably 0.05% to 7.5%, most preferably 0.1% to 5% by weight of the total composition.

Conditioners of the invention will typically also incorporate a fatty alcohol. The combined use of fatty alcohols and cationic surfactants in conditioning compositions is believed to be especially advantageous, because this leads to the formation of a lamellar phase, in which the cationic surfactant is dispersed.

Representative fatty alcohols comprise from 8 to 22 carbon atoms, more preferably 16 to 22. Fatty alcohols are typically compounds containing straight chain alkyl groups. Examples of suitable fatty alcohols include cetyl alcohol, stearyl alcohol and mixtures thereof. The use of these materials is also advantageous in that they contribute to the overall conditioning properties of compositions of the invention.

The level of fatty alcohol in conditioners of the invention will generally range from 0.01% to 10%, preferably from 0.1% to 8%, more preferably from 0.2% to 7%, most preferably from 0.3% to 6% by weight of the composition. The weight ratio of cationic surfactant to fatty alcohol is suitably from 1:1 to 1:10, preferably from 1:1.5 to 1:8, optimally from 1:2 to 1:5. If the weight ratio of cationic surfactant to fatty alcohol is too high, this can lead to eye irritancy from the composition. If it is too low, it can make the hair feel squeaky for some consumers.

Adjuncts

The compositions of the present invention may also contain adjuncts suitable for hair care. Generally such ingredients are included individually at a level of up to 2 wt %, preferably up to 1 wt % of the total composition.

Suitable hair care adjuncts, include perfumes; amino acids, sugars and ceramides and viscosity modifiers.

Product Form

Compositions of the present invention are formulated into hair colouring compositions which may take a variety of forms, including, for example, mousses, gels, lotions, creams, sprays and tonics. These product forms are well known in the art.

The preferred product is a lotion, cream, mousse or gel.

Preferably, the composition is a rinse off hair treatment composition. A rinse off composition is applied hair preferably to wet hair, and left on the hair for up to 1 hour, preferably left on the hair for up to 30 minutes before it is removed by rinsing.

Example of the invention will be illustrated with the following non-limiting Examples.

Examples of the invention are illustrated by a number, comparative Examples by a letter.

### EXAMPLES

### Example 1

Polymer A (control): 40 g of a branched polyethylene imine (pH˜11) with a M_{w} of 1300 was mixed with 20 g of reactive red 239 in 260 ml of water and stirred at 55° C. for 2 hours, then pH adjusted to 7 with HCl.

Polymer 1: 40 g of a branched polyethylene imine (pH˜11) with a M_{w} of 1300 was mixed 4 g of styrene oxide and stirred for 18 hours at room temperature. 20 g of reactive red 239 was then added and stirred at 55° C. for 2 hours, then pH adjusted to 7 with HCl.

A 50:50 grey human hair swatch weighing 0.7 g was placed in an aqueous solution at pH=9, containing 0.15 wt % of dye-polymer. The solution was agitated for 5 minutes at room temperature and the hair swatch removed, rinsed under running water and dried. The colour of the swatch was then measured using a reflectometer and expressed as the CIE L*a*b* values. The dye-polymers gave an attractive red hue to the hair, and this was measured as the increase in a* value relative to an undyed swatch:

Δa=a(dyed)−a(undyed)

The relative substantivity of the polymers was calculated as Δa/Δa(polymer A) and the results shown below

| | substantivity |
|---|---|
| Polymer A (reference) | 1 |
| Polymer 1 (arylated) | 3.1 |

The arylated polymer give greater substantivity.

### Example 2

Polymer B (control): 40 g of a branched polyethylene imine (pH˜11) with a M_{w} of 2000 was mixed with 10 g of reactive blue 198 in 260 ml of water and stirred at room temperature for 18 hours, then pH adjusted to 7 with HCl. The crude product was dialysed against deionized water.

Polymer 2: 40 g of a branched polyethylene imine (pH˜11) with a M_{w} of 2000 was mixed 10 g of reactive blue 198 in 260 ml of water and stirred for 18 hours at room temperature. The pH of PEI-Dye was adjusted to 7 with HCl. 10 g of 3,4-dihydroxybenzonic acid was mixed with 1-ethyl-3-[3-dimethylaminopropyl]carbodiimide hydrochloride, 15 g, and 2-N-hydroxysuccinimide, 9 g, dispersed in 100 g water, and then added into PEI-Dye solution. The solution was kept at pH 5, and stirred at room temperature for 18 hours. The crude product was dialysed against deionized water.

A bleached white chinese human hair swatch weighing 0.7 g was placed in an aqueous solution at pH=9, containing 0.15 wt % of dye-polymer. The solution was agitated for 5 minutes at room temperature and the hair swatch removed, rinsed under running water and dried. The colour of the swatch was then measured using a reflectometer and expressed as the CIE L*a*b* values. The dye-polymers gave a dark blue shade to the grey hair, and this was measured as the decrease in L* value relative to an undyed swatch:

ΔL=L(dyed)−L(undyed)

The relative substantivity of the polymers was calculated as ΔL/ΔL(polymer A) and the results shown below:

| | substantivity |
|---|---|
| Polymer B (control) | 1 |
| Polymer 2 (arylated) | 2.2 |

The arylated polymer give greater substantivity.

### Example 3

Polymer C: 40 g of a branched polyethylene imine (pH˜11) with a M_{w} of 2000 was mixed with 10 g of reactive blue 49 in 260 ml of water and stirred at room temperature for 18 hours, then pH adjusted to 7 with HCl. The crude product was dialysed against deionized water and lyophilized.

Polymer 3: 40 g of a branched polyethylene imine (pH˜11) with a M_{w} of 2000 was mixed 4 g of styrene oxide and stirred for 18 hours at room temperature. 10 g of reactive blue 49 was then added and stirred at 55° C. for 2 hours, then pH adjusted to 7 with HCl. The crude product was dialysed against deionized water and lyophilized.

Polymer 3 and C were assessed for their skin staining potential from a conditioner formulation. A hair conditioner was produced by a standard process

| Ingredient | wt % |
|---|---|
| Water | To 100 |
| Lactic acid | 0.38 |
| Methyl parahydroxybenzoate | 0.2 |
| Stearyl alcohol | 5 |
| Behenyltrimmonium chloride | 0.87 |
| LexamineS-13 (100% TAS) | 1.25 |
| Silicone emulsion | 2.5 |
| Perfume | 0.5 |
| Dye-polymer | 2 |

The following protocol was used:
1. Prepare 3 strips of pig skin 6.0 cm×2.0 cm per treatment
2. Measure baseline Lab levels and photograph
3. Apply 0.15 g of shampoo and gently massage onto skin for 30 secs (as would be case in vivo)
4. Incubate at RT for 90 sec
5. Rinse for 30 seconds under running tap water (40 oC)
6. Dab with tissue to remove excess water
7. Apply 0.15 g of conditioner and gently massage onto skin for 60 secs (as would be case in vivo)
8. Incubate at RT for 180 sec
9. Rinse for 60 seconds under running tap water (40 oC)
10. Dab with tissue to remove excess water.
11. Measure Lab levels & photograph.
12. Repeat 3-6, and measure Lab levels & photograph.

The colour of the pig skin was then measured using a reflectometer and expressed as the CIE L*a*b* values. The dye-polymers gave a dark blue shade to the skin when excessive deposition had occurred. This was measured as the change in L* value relative to an undyed pig skin:

ΔL=L(dyed)−L(undyed)

A polymeric dye with low skin staining will have the smallest change in L* verses the untreated pig skin.

| | Change in L* verses undyed skin |
|---|---|
| Polymer C (reference) | 23.5 |
| Polymer 3 (arylated) | 17.1 |

1. A hair colouring composition comprising an arylated polyethyleneimine dye wherein the arylated polyethyleneimine dye comprises a polyethyleneimine having covalently bound to the nitrogen groups of the polyethyleneimine:
a. a negatively charged reactive dye and;
b. aryl group or substituted aryl group;
c. in which the aryl group or substituted aryl group and the negatively charged reactive dye are different moieties.

2. A hair colouring composition according to claim 1 in which the polyethyleneimine of the arylated polyethyleneimine dye has from 6 to 100 nitrogen atoms.

3. A hair colouring composition according to claim 2 in which the polyethyleneimine of the arylated polyethyleneimine dye has from 12 to 40 nitrogen atoms.

4. A hair colouring composition according to claim 1 in which the reactive dye of the arylated polyethyleneimine dye comprises a reactive group selected from dichlorotriazinyl, difluorochloropyrimidine, monofluorotrazinyl, dichloroquinoxaline, vinylsulfone, difluorotriazine, monochlorotriazinyl, bromoacrlyamide and trichloropyrimidine.

5. A hair colouring composition according to claim 1 in which the reactive dye of the arylated polyethyleneimine dye comprises a chromophore selected from azo, anthraquinone, phthalocyanine, formazan and triphendioxazine.

6. A hair colouring composition according to claim 1 in which the reactive dye of the arylated polyethyleneimine dye is sulphonated.

7. A hair colouring composition according to claim 1 in which mole ratio of PEI to dye is from 1:1 to 2:1.

8. A hair colouring composition according to claim 1 in which the substituted aryl group of the arylated polyethyleneimine dye has the structure: 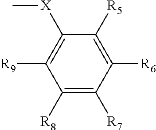
in which X is a hydroxy amide or amide group, R₅, R₆, R₇, R₈, R₉ are independently selected from H, C1-C1 alkyl or hydroxy groups, such that at least two R₁ to R₅ groups are equal to H.

9. A hair colouring composition according to claim 8 in which the substituted aryl group of the arylated polyethyleneimine dye is phenyl.

10. A hair colouring composition according to claim 1 in which the level of arylated polyethyleneimine dye is from 0.01 to 5 wt %.

11. Method for dying hair comprising the steps of applying to the hair a composition as described in claim 1.

12. Use of an arylated polyethylenimine dye described in claim 1 for colouring the hair.

13. A polyethyleneimine having covalently bound to the nitrogen groups of the polyethyleneimine:
(i) a negatively charged reactive dye and;
(ii) aryl group or substituted aryl group;
in which the aryl group or substituted aryl group and the negatively charged reactive dye are different moieties.
